(19) United States
(12) Patent Application Publication
(10) Pub. No.: US 2012/0077837 A1
(43) Pub. Date: Mar. 29, 2012

(54) ANTI-TUMOR AGENT

(75) Inventors: Kiyoshi Okamoto, Tsukuba (JP); Yoichi Ozawa, Tsukuba (JP)

(73) Assignee: EISAI R&D MANAGEMENT CO., LTD., Tokyo (JP)

(21) Appl. No.: 13/238,085

(22) Filed: Sep. 21, 2011

Related U.S. Application Data
(60) Provisional application No. 61/386,333, filed on Sep. 24, 2010.

(51) Int. Cl. A61K 31/436; A61P 35/00

(52) U.S. Cl. 514/291

(57) ABSTRACT

A combination therapy for tumor is provided. Disclosed is a method of treating tumor, the method comprises administering to a patient, a compound or pharmaceutically acceptable salt thereof represented by Formula (I) and everolimus represented by Formula (II):

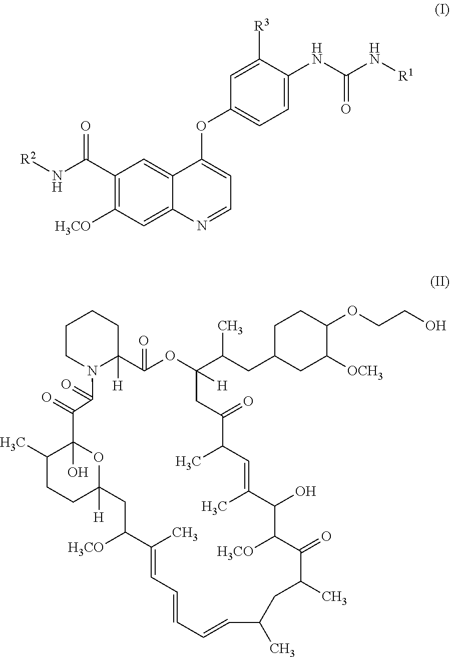

wherein R¹ is C₁₋₆ alkyl or C₃₋₈ cycloalkyl; R² is a hydrogen atom or C₁₋₆ alkoxy; and R³ is a hydrogen atom or a halogen atom.

### CROSS-REFERENCE TO RELATED APPLICATIONS

This application claims the benefit of U.S. Provisional Application No. 61/386,333 filed on Sep. 24, 2010, the entire content of which is incorporated herein by reference.

### BACKGROUND OF THE INVENTION

1. Field of the Invention

The present invention relates to a method of treating tumor by a combination of a compound having a kinase-inhibitory action and everolimus. More particularly, the present invention relates to a method of treating tumor by a combination of a compound having a multi-tyrosine kinase inhibitory action and a compound having a mammalian target of rapamycin (mTOR) protein-inhibitory action.

2. Related Background Art

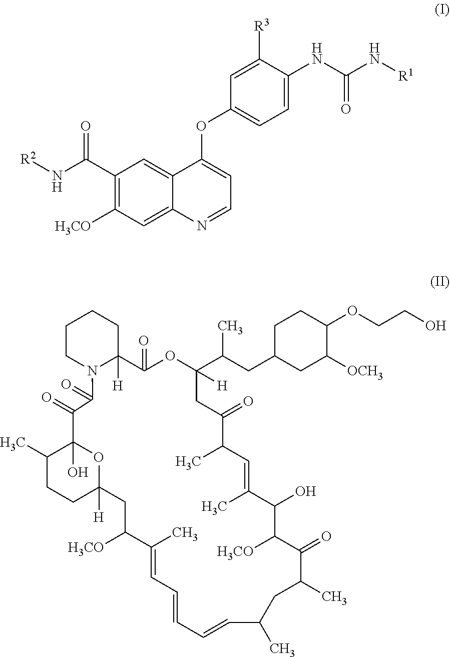

wherein R¹ is C₁₋₆ alkyl or C₃₋₈ cycloalkyl; R² is a hydrogen atom or C₁₋₆ alkoxy; and R³ is a hydrogen atom or a halogen atom.

A compound represented by Formula (I) has anti-angiogenic actions (Patent Document 1), inhibitory actions (Patent Documents 2 to 5) against tyrosine kinases which are reported to be involved in malignant alteration of tumors (Non-patent Documents 1 to 5), and the like; and is known as a therapeutic agent for various tumors such as thyroid cancer, lung cancer, melanoma, endometrial cancer, gastric cancer and bladder cancer.

On the other hand, the compound represented by Formula (II) is a derivative of rapamycin which is known as a macrolide antibiotic. This compound is referred to as everolimus (42-O-(2-hydroxyethyl)-rapamycin), and it has been reported that the compound may be used as an antitumor agent (Patent Document 6, Non-patent Document 6) or as a therapeutic agent for autoimmune diseases (Patent Document 7).

In general, antitumor agents are often not effective for all of the patients when they were used individually. Thus, attempts have been made so far to increase the cure rate by combination of plural antitumor agents (Patent Documents 8 to 10).

### LIST OF REFERENCES

Patent Document 1: WO 2002/032872
Patent Document 2: WO 2004/080462
Patent Document 3: WO 2007/015569
Patent Document 4: WO 2007/136103
Patent Document 5: WO 2008/026748
Patent Document 6: WO 2002/066019
Patent Document 7: WO 1994/09010
Patent Document 8: WO 2009/140549
Patent Document 9: US Patent Application Publication 2004-259834
Patent Document 10: U.S. Pat. No. 6,217,866
Non-patent Document 1: Lasota et al., “Mutations in Exons 9 and 13 of KIT Gene Are Rare Events in Gastrointestinal Stromal Tumors”, American Journal of Pathology, 157, 1091-1095, 2000.
Non-patent Document 2: Berdel et al., “Recombinant Human Stem Cell Factor Stimulates Growth of a Human Glioblastoma Cell Line Expressing c-kit Protooncogenel”, Cancer Research, 52, 3498-3502, 1992.
Non-patent Document 3: Lennartsson et al., “The stem cell factor receptor/c-Kit as a drug target in cancer”, Current Cancer Drug Targets, 6, 65-75, 2006.
Non-patent Document 4: Turner et al., “Fibroblast growth factor signalling: from development to cancer”, Nature Reviews, Cancer, 10, 116-129, 2010.
Non-patent Document 5: Wells et al., “Targeting the RET Pathway in Thyroid Cancer”, Clinical Cancer Research, 15, 7119-7123, 2009.
Non-patent Document 6: Di Lorenzo et al., “Targeted Therapy in the Treatment of Metastatic Renal Cell Cancer”, Oncology, 77 (Suppl. 1), 122-131, 2009.

### SUMMARY OF THE INVENTION

However, the therapeutic effects, which have been reported so far, obtained by combination of plural antitumor agents were insufficient, and hence development of a novel combination therapy using antitumor agents has been expected.

In view of such circumstances, the present inventors intensively studied to discover that administration of a combination of the compounds represented by Formula (I) and Formula (II) to a patient suffering from a tumor attains an unexpectedly excellent antitumor effect, thereby completing the present invention.

That is, the present invention provides [1] to [8] below.

[1] An antitumor agent for combined use of:

a compound or pharmaceutically acceptable salt thereof represented by Formula (I):

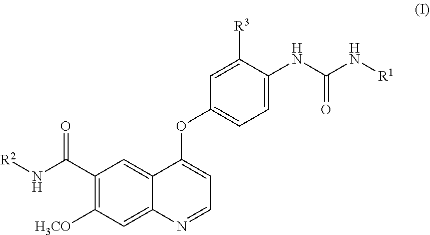

wherein R¹ is C₁₋₆ alkyl or C₃₋₈ cycloalkyl; R² is a hydrogen atom or C₁₋₆ alkoxy; and R³ is a hydrogen atom or a halogen atom
and

everolimus represented by Formula (II):

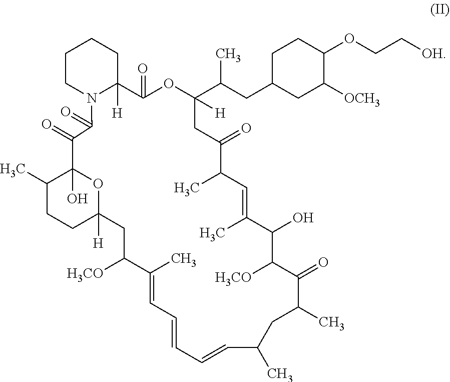

[2] An antitumor agent for simultaneous or separate administration of a compound or pharmaceutically acceptable salt thereof represented by the above Formula (I), and everolimus represented by the above Formula (II).
[3] An antitumor agent comprising a compound or pharmaceutically acceptable salt thereof represented by the above Formula (I), and everolimus represented by the above Formula (II).
[4] Everolimus represented by the above Formula (II) for therapy of a tumor by combined use with a compound or pharmaceutically acceptable salt thereof represented by the above Formula (I).
[5] A compound or pharmaceutically acceptable salt thereof represented by the above Formula (I) for therapy of a tumor by combined use with everolimus represented by the above Formula (II).
[6] A method of treating a tumor, wherein a compound or pharmaceutically acceptable salt thereof represented by the above Formula (I), and everolimus represented by the above Formula (II) are used in combination.
[7] A pharmaceutical composition comprising a compound or pharmaceutically acceptable salt thereof represented by the above Formula (I) everolimus represented by the above Formula (II), and a vehicle.
[8] A kit comprising:

a pharmaceutical composition comprising a compound or pharmaceutically acceptable salt thereof represented by the above Formula (I) and a vehicle; and

a pharmaceutical composition comprising everolimus represented by the above Formula (II), and a vehicle.

The compound represented by the above Formula (I) is preferably one or more compounds selected from the group consisting of 4-[3-chloro-4-(cyclopropylaminocarbonyl)aminophenoxy]-7-methoxy-6-quinolinecarboxamide:

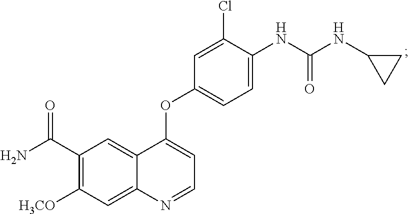

4-[3-chloro-4-(methylaminocarbonyl)aminophenoxy]-7-methoxy-6-quinolinecarboxamide:

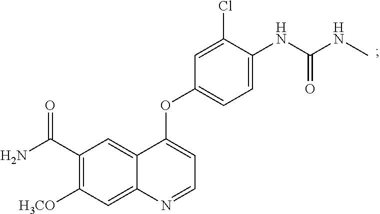

4-[3-chloro-4-(ethylaminocarbonyl)aminophenoxy]-7-methoxy-6-quinolinecarboxamide:

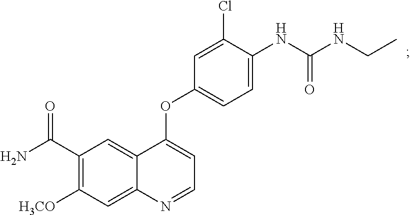

N6-methoxy-4-(3-chloro-4-{[(cyclopropylamino)carbonyl)amino]phenoxy}-7-methoxy-6-quinolinecarboxamide:

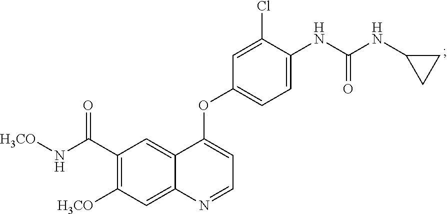

and
N6-methoxy-4-(3-chloro-4-{[(ethylamino)carbonyl]amino}phenoxy)-7-methoxy-6-quinolinecarboxamide:

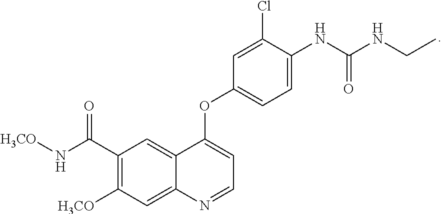

The compound represented by the above Formula (I) is more preferably 4-[3-chloro-4-(cyclopropylaminocarbonyl)aminophenoxy]-7-methoxy-6-quinolinecarboxamide:

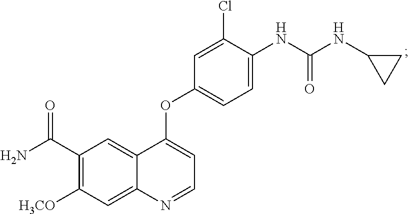

and this compound may be hereinafter referred to as Compound A.

The present invention provides a method of treating tumor by a combination of a compound having a multi-tyrosine kinase inhibitory action and everolimus. Such a method shows an excellent antitumor effect compared to cases where these are individually used, and shows antitumor effects against various cancer types.

### BRIEF DESCRIPTION OF THE DRAWINGS

FIG. 1 is a graph showing a combined effect of Compound A and everolimus in a model animal to which human renal cell carcinoma cell line (Caki-1) was transplanted.

### DESCRIPTION OF THE PREFERRED EMBODIMENTS

The compound or pharmaceutically acceptable salt thereof represented by Formula (I) according to the present invention can be produced by the method described in Patent Document 1. Further, everolimus represented by Formula (II) according to the present invention can be produced by the method described in Patent Document 7.

Examples of the pharmaceutically acceptable salt include salts with inorganic acids, salts with organic acids, salts with inorganic bases, salts with organic bases, and salts with acidic or basic amino acids.

Preferred examples of the salts with inorganic acids include salts with hydrochloric acid, hydrobromic acid, sulfuric acid, nitric acid, phosphoric acid and the like. Preferred examples of the salts with organic acids include salts with acetic acid, succinic acid, fumaric acid, maleic acid, tartaric acid, citric acid, lactic acid, stearic acid, benzoic acid, methanesulfonic acid, ethanesulfonic acid, p-toluenesulfonic acid and the like.

Preferred examples of the salts with inorganic bases include alkaline metal salts such as a sodium salt and a potassium salt; alkaline earth metal salts such as a calcium salt and a magnesium salt; an aluminum salt; and an ammonium salt. Preferred examples of the salts with organic bases include salts with diethylamine, diethanolamine, meglumine, N,N-dibenzylethylenediamine and the like.

Preferred examples of the salts with acidic amino acids include salts with aspartic acid, glutamic acid and the like. Preferred examples of the salts with basic amino acids include salts with arginine, lysine, ornithine and the like.

Especially preferred pharmaceutically acceptable salts are salts with organic acids.

The antitumor agent of the present invention may be orally administered in the form of a solid formulation such as a tablet, granule, fine granule, powder or capsule, or in the form of a liquid, jelly, syrup or the like.

Further, the antitumor agent of the present invention may be parenterally administered in the form of an injection, suppository, ointment, cataplasm or the like.

The dose of the compound or pharmaceutically acceptable salt thereof represented by Formula (I) may be appropriately selected depending on the degrees of symptoms, age, sex and body weight of the patient, difference in sensitivity, route, time and interval of administration, type of pharmaceutical formulation, and/or the like. Usually, in cases where oral administration is carried out for an adult (60 kg body weight), the dose is 1 to 600 mg, preferably 5 to 400 mg, more preferably 5 to 200 mg per day. This may be administered at one time, or dividedly at 2 or 3 times per day.

The dose of everolimus represented by Formula (II) may be appropriately selected as in the case described above. Usually, in cases where oral administration is carried out for an adult (60 kg body weight), the dose is 1 to 50 mg, preferably 1 to 20 mg, more preferably 5 to 10 mg per day. This may be administered at one time, or dividedly at 2 or 3 times per day.

In cases where an oral solid formulation is prepared, a vehicle, and, as required, a binder, disintegrator, lubricant, coloring agent, flavoring agent and/or the like may be added to the principal component, that is, a compound or pharmaceutically acceptable salt thereof represented by Formula (I), and everolimus represented by Formula (II), to prepare, thereafter, a tablet, granule, fine granule, powder, capsule or the like according to a conventional method.

Examples of the vehicle include lactose, corn starch, white soft sugar, glucose, sorbitol, crystalline cellulose and silicon dioxide. Examples of the binder include polyvinyl alcohol, ethylcellulose, methylcellulose, gum Arabic, hydroxypropylcellulose and hydroxypropylmethylcellulose. Examples of the lubricant include magnesium stearate, talc and silica. Examples of the coloring agent include titanium oxide, iron sesquioxide, yellow iron sesquioxide, cochineal, carmine and riboflavin. Examples of the flavoring agent include cocoa powder, ascorbic acid, tartaric acid, peppermint oil, borneol and cinnamon powder. These tablets and granules may be coated as required.

In cases where an injection is prepared, a pH adjustor, buffering agent, suspending agent, solubilizer, stabilizer, isotonic agent, preservative and/or the like may be added as required to the principal component, to prepare an intravenous, subcutaneous or intramuscular injection, or an intravenous drip infusion. As required, these may be prepared into lyophilized products by conventional methods.

Examples of the suspending agent include methylcellulose, polysorbate 80, hydroxyethylcellulose, gum Arabic, powdered tragacanth, sodium carboxymethylcellulose and polyoxyethylene sorbitan monolaurate.

Examples of the solubilizer include polyoxyethylene hydrogenated castor oil, polysorbate 80, nicotinamide, polyoxyethylene sorbitan monolaurate, macrogol and glycerin fatty acid ester.

Examples of the stabilizer include sodium sulfite and sodium metabisulfite. Examples of the preservative include methyl parahydroxybenzoate, ethyl parahydroxybenzoate, sorbic acid, phenol, cresol and chlorocresol.

The antitumor agent of the present invention may be prepared by formulating a compound or pharmaceutically acceptable salt thereof represented by Formula (I), and everolimus represented by Formula (II) separately, and the both may be administered either at the same time or separately. Further, the two formulations may be placed in a single package, to provide the so called kit formulation. Further, the both compounds may be contained in a single formulation.

The type of the tumor to be treated with the antitumor agent of the present invention is not restricted, and examples thereof include fibroma, adipoma, myxoma, chondroma, osteoma, angioma, lymphoma, myeloma, melanoma, myoma, neuroma, glioma, schwannoma, sarcoma, osteosarcoma, myosarcoma, fibrosarcoma, papilloma, adenoma, cystoma, brain tumor, cancers such as cervical cancer, esophagus cancer, tongue cancer, lung cancer, breast cancer, pancreatic cancer, gastric cancer, small intestinal cancer in duodenum, jejunum, ileum and the like, large bowel cancer in colon, caecum, rectum and the like, bladder cancer, renal cancer, liver cancer, gallbladder cancer, prostate cancer, uterine cancer, ovarian cancer, thyroid cancer and pharyngeal cancer; and mixed tumors thereof.

### EXAMPLES

The present invention is described in more detail by way of Examples below.

### Example 1

### Tumor Growth-Inhibitory Action by Combined Use of Compound a and Everolimus in Human Renal Cell Carcinoma Cell Line (Caki-1)

Using 5 individuals per group of nude mice (CAnN.Cg-Foxn1nu/Cr1Cr1j, female, Charles River Japan, Inc.), the antitumor effect obtained when Compound A, everolimus or the both compounds was/were administered was evaluated. Human-derived renal cell carcinoma cell line Caki-1(ATCC) was added to a medium (RPMI-1640, Wako Pure Chemical Industries, Ltd.) to a density of 2×10⁸ cells/mL and suspended therein. To the resulting suspension, an equal volume of Matrigel™ matrix (Nippon Becton Dickinson Co., Ltd.) was added, and the resulting mixture was mixed sufficiently. The mixture was subcutaneously transplanted at the left flank of each mouse in an amount of 0.1 mL.

Forty eight days after the transplantation, the major axis and the minor axis were measured using an electronic digital caliper (Digimatic™ caliper, Mitutoyo Corporation). The mice were separated into groups such that the average value of the tumor volume is almost the same among the groups. The volume of the tumor was calculated according to the equation below.

Tumor volume=major axis (mm)×minor axis (mm)×minor axis (mm)/2

Compound A was dissolved in purified water to a concentration of 1 mg/mL. Further, everolimus was suspended in a mixed solution of DMSO, Tween 80 and glucose (3.5:6.5:90 (V/V/V), hereinafter referred to as solvent) to a concentration of 3 mg/mL.

To the mice of each group, the Compound A solution and the solvent, the everolimus suspension and purified water, or the Compound A solution and the everolimus suspension were orally administered in an amount of 10 mL/kg once per day for 15 days. The doses of Compound A and everolimus were 10 mg/kg and 30 mg/kg, respectively. To the control group, 10 mL/kg each of purified water and the solvent was administered.

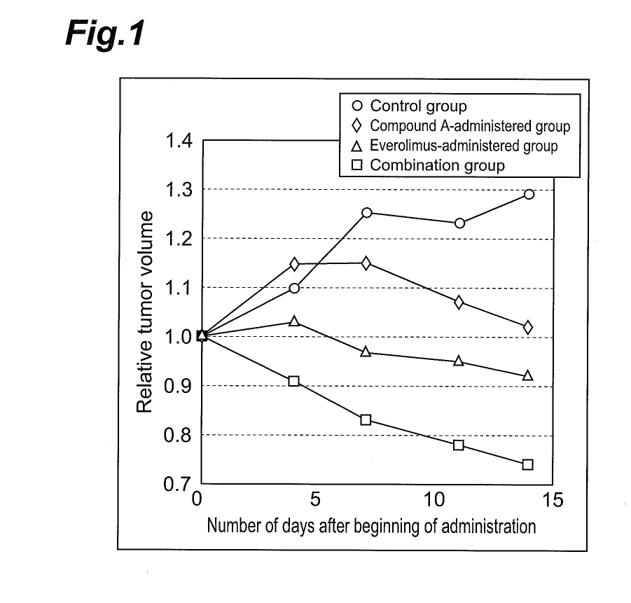

The tumor volume of each mouse was measured on Day 4, 7, 11 and 14 after the beginning of the administration. The ratio of the tumor volume observed on each day of the measurement relative to the tumor volume observed on the day when the administration was begun was calculated as the relative tumor volume (hereinafter referred to as RTV). The results are shown in Table 1 and FIG. 1.

TABLE 1

| | Day 4 | Day 7 | Day 11 | Day 14 |
|---|---|---|---|---|
| Control group | 1.10 | 1.25 | 1.23 | 1.29 |
| Compound A | 1.15 | 1.15 | 1.07 | 1.02 |
| group | | | | |
| Everolimus group | 1.03 | 0.97 | 0.95 | 0.92 |
| Combination | 0.91 | 0.83 | 0.78 | 0.74 |
| group | | | | |

What is claimed is:

1. A method of treating tumor comprising administering to a patient,
a compound or pharmaceutically acceptable salt thereof represented by Formula (I): 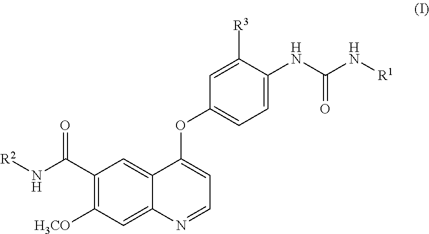
wherein R¹ is C₁₋₆ alkyl or C₃₋₈ cycloalkyl; R² is a hydrogen atom or C₁₋₆ alkoxy; and R³ is a hydrogen atom or a halogen atom and
everolimus represented by Formula (II):

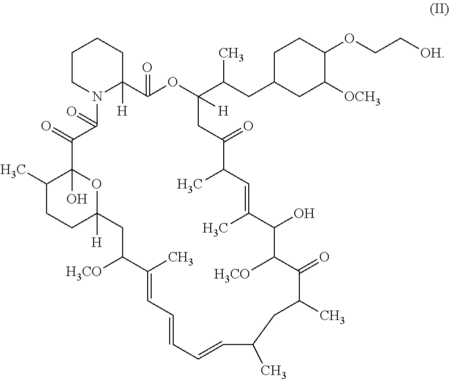

2. The method according to claim 1, wherein the compound or pharmaceutically acceptable salt thereof represented by Formula (I) and everolimus is administered simultaneously.

3. The method according to claim 1, wherein the compound or pharmaceutically acceptable salt thereof represented by Formula (I) and everolimus is administered separately.

4. The method according to claim 1, wherein a pharmaceutical composition comprising the compound or pharmaceutically acceptable salt thereof represented by Formula (I), everolimus and a vehicle is administered.

5. The method according to claim 1, wherein a pharmaceutical composition comprising the compound or pharmaceutically acceptable salt thereof represented by Formula (I) a vehicle and a pharmaceutical composition comprising everolimus and a vehicle is administered.

6. The method according to claim 1, wherein the compound represented by Formula (I) is one or more of compounds selected from the group consisting of:
4-[3-chloro-4-(cyclopropylaminocarbonyl)aminophenoxy]-7-methoxy-6-quinolinecarboxamide: 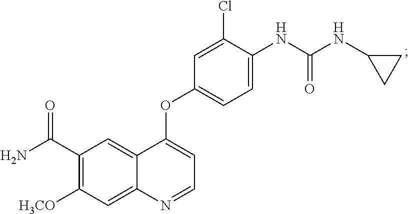
4-[3-chloro-4-(methylaminocarbonyl)aminophenoxy]-7-methoxy-6-quinolinecarboxamide: 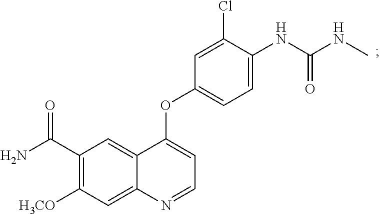
4-[3-chloro-4-(ethylaminocarbonyl)aminophenoxy]-7-methoxy-6-quinolinecarboxamide: 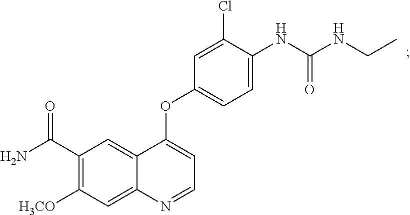
N6-methoxy-4-(3-chloro-4-{[(cyclopropylamino)carbonyl)amino]phenoxy}-7-methoxy-6-quinolinecarboxamide: 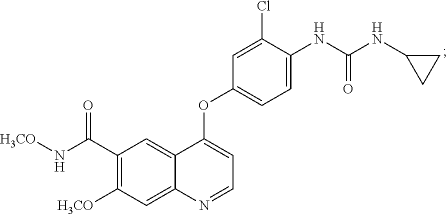
and
N6-methoxy-4-(3-chloro-4-[(ethylamino)carbonyl]amino phenoxy)-7-methoxy-6-quinolinecarboxamide:

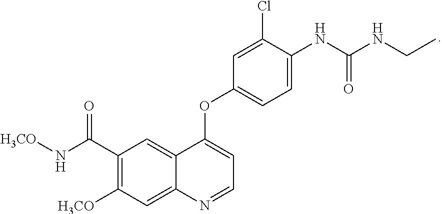

7. The method according to claim 1, wherein the compound represented by Formula (I) is 4-[3-chloro-4-(cyclopropylaminocarbonyl)aminophenoxy]-7-methoxy-6-quinolinecarboxamide:

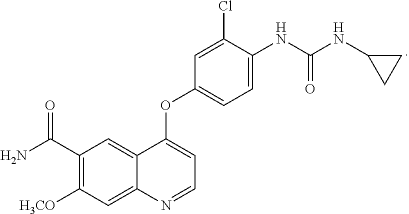

8. A pharmaceutical composition comprising a compound or pharmaceutically acceptable salt thereof represented by Formula (I): 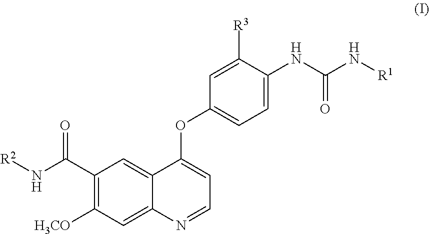
wherein R¹ is C₁₋₆ alkyl or C₃₋₈ cycloalkyl; R² is a hydrogen atom or C₁₋₆ alkoxy; and R³ is a hydrogen atom or a halogen atom,
everolimus represented by Formula (II): 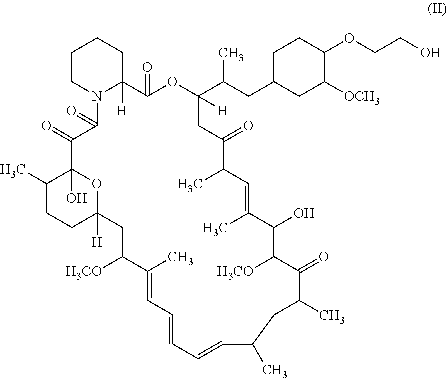
and a vehicle.

9. The pharmaceutical composition according to claim 8, wherein the compound represented by Formula (I) is one or more of compounds selected from the group consisting of:
4-[3-chloro-4-(cyclopropylaminocarbonyl)aminophenoxy]-7-methoxy-6-quinolinecarboxamide: 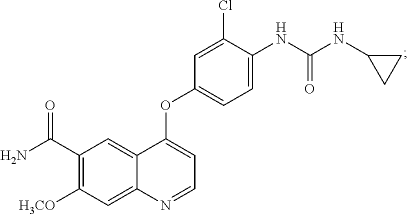
4-[3-chloro-4-(methylaminocarbonyl)aminophenoxy]-7-methoxy-6-quinolinecarboxamide: 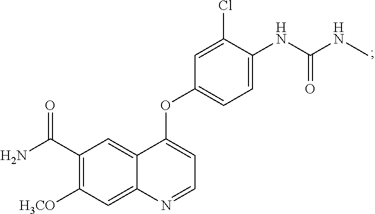
4-[3-chloro-4-(ethylaminocarbonyl)aminophenoxy]-7-methoxy-6-quinolinecarboxamide: 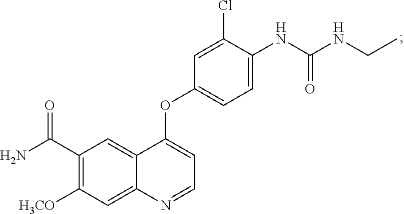
N6-methoxy-4-(3-chloro-4-{[(cyclopropylamino)carbonyl)amino]phenoxy}-7-methoxy-6-quinolinecarboxamide: 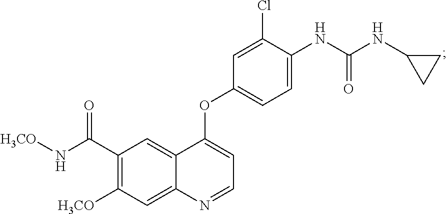
and
N6-methoxy-4-(3-chloro-4-{[(ethylamino)carbonyl]amino}phenoxy)-7-methoxy-6-quinolinecarboxamide:

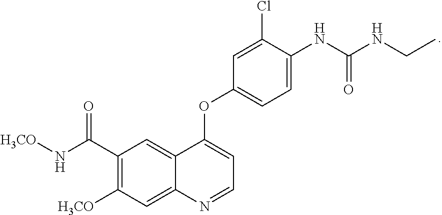

10. The pharmaceutical composition according to claim 8, wherein the compound represented by Formula (I) is 4-[3-chloro-4-(cyclopropylaminocarbonyl)aminophenoxy]-7-methoxy-6-quinolinecarboxamide:

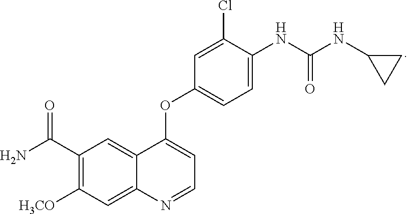

11. A kit comprising:
a pharmaceutical composition comprising a compound or pharmaceutically acceptable salt thereof represented by Formula (I): 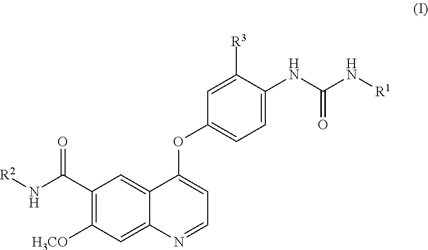
wherein R¹ is C₁₋₆ alkyl or C₃₋₈ cycloalkyl; R² is a hydrogen atom or C₁₋₆ alkoxy; and R³ is a hydrogen atom or a halogen atom,
and a vehicle; and
a pharmaceutical composition comprising everolimus represented by Formula (II): 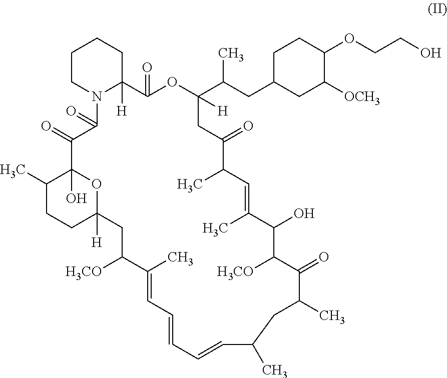
and a vehicle.

12. The kit according to claim 11, wherein the compound represented by Formula (I) is one or more of compounds selected from the group consisting of:
4-[3-chloro-4-(cyclopropylaminocarbonyl)aminophenoxy]-7-methoxy-6-quinolinecarboxamide: 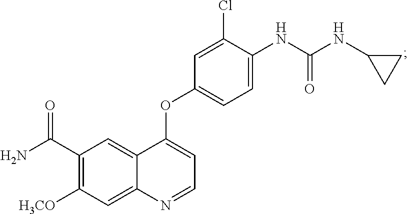
4-[3-chloro-4-(methylaminocarbonyl)aminophenoxy]-7-methoxy-6-quinolinecarboxamide: 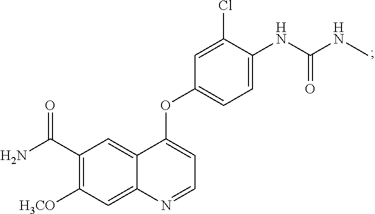
4-[3-chloro-4-(ethylaminocarbonyl)aminophenoxy]-7-methoxy-6-quinolinecarboxamide: 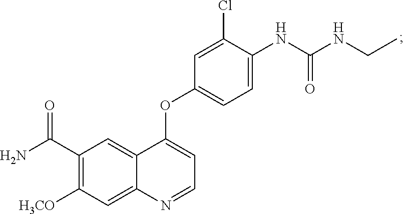
N6-methoxy-4-(3-chloro-4-{[(cyclopropylamino)carbonyl)amino]phenoxy}-7-methoxy-6-quinolinecarboxamide: 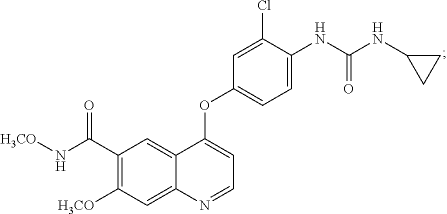
and
N6-methoxy-4-(3-chloro-4-{[(ethylamino)carbonyl]amino}phenoxy)-7-methoxy-6-quinolinecarboxamide:

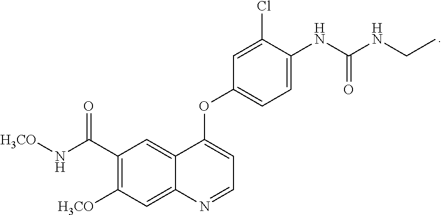

13. The kit according to claim 11, wherein the compound represented by Formula (I) is 4-[3-chloro-4-(cyclopropylaminocarbonyl)aminophenoxy]-7-methoxy-6-quinolinecarboxamide: [chemical structure]
